(19) United States
(12) Patent Application Publication
(10) Pub. No.: US 2025/0049496 A1
(43) Pub. Date: Feb. 13, 2025

(54) HANDLING DEVICE FOR AN ELECTROSURGICAL INSTRUMENT, METHOD FOR PRODUCING A HANDLING DEVICE, AND ELECTROSURGICAL INSTRUMENT

(71) Applicant: KARL STORZ SE & Co. KG, Tuttlingen (DE)

(72) Inventors: Janosz SCHNEIDER, Tuttlingen (DE); Jochen STEFAN, Tuttlingen (DE)

(21) Appl. No.: 18/717,501

(22) PCT Filed: Nov. 30, 2022
(86) PCT No.: PCT/EP2022/083813

§ 371 Date: Jun. 7, 2024

(30) Foreign Application Priority Data
Dec. 9, 2021 (DE) 10 2021 132 425.4

(51) Int. Cl. A61B 18/14; A61B 18/00

(52) U.S. Cl. CPC A61B 18/1445; A61B 2018/00083

(57) ABSTRACT

A handling device for an electro-surgical instrument includes a joint mechanism which has at least one handling-side lever which can be moved for manipulation; a lever extension which is configured as an electrical insulator and is fixedly coupled to the lever; and an electrically conductive contact portion which is fixedly coupled to the lever extension. The electrically conductive contact portion is arranged on a side of the lever extension which can be subjected to force via the joint mechanism and is configured for electro-surgical contacting biological tissue. A method produces a handling device for an electro-surgical instrument and a corresponding electro-surgical instrument, in particular an endoscopic instrument.

### CROSS REFERENCE TO RELATED APPLICATIONS

This application is a United States National Phase Application of International Application PCT/EP2022/083813, filed Nov. 30, 2022, and claims the benefit of priority under 35 U.S.C. § 119 of German Application 10 2021 132 425.4, filed Dec. 9, 2021, the entire contents of which are incorporated herein by reference.

### TECHNICAL FIELD

The present invention pertains to a handling device for an electro-surgical instrument, a method for producing a handling device for an electro-surgical instrument, and a corresponding electro-surgical instrument, in particular an endoscopic instrument.

### BACKGROUND

Handling devices for surgical instruments, also known as accessories, must meet strict biocompatibility requirements and are usually made of steel. They usually contain two lever arms and a joint mechanism that allows one or both levers to be moved. There are corresponding handling devices for hand-operated surgical instruments and also handling devices for robot-operated surgical instruments. Both, hand-operated surgical instruments and robot-operated surgical instruments, are designed in particular as endoscopic instruments.

EP 2 026 705 B1 describes a gripping forceps for hand-operated surgical instruments, which has two gripping jaws that can move relative to a main body. This design offers high mechanical stability and strong tractive force transmission, but it is not an electrically active instrument.

DE 10 2019 121 034 A1 describes an endoscopic device intended for a surgical robot system with a deflection mechanism and an end effector coupled to it in the form of forceps.

Current surgical techniques, in particular so-called HF surgery, use high-frequency alternating electric current for electrotomy (also known as diathermy or electrocaustic surgery) and coagulation (clotting). The thermal effect of high-frequency alternating current on tissue is used to cut through target tissue and/or stop bleeding. The surgical instruments have one or more (active) electrodes on their handling device, e.g., forceps, clamps, scissors or the like, via which the high-frequency alternating current is introduced into the target tissue of a patient. To achieve the desired effect of electrotomy and/or coagulation on the target tissue, a high current density is applied to the target tissue. For this reason, (active) electrodes generally have a small surface area and are shaped like needles or blades, for example, in order to introduce the applied high-frequency alternating current into the target tissue with the highest possible current density. The handling devices are correspondingly small.

With the previous systems, the alternating current was conducted to the handling device either via electrically conductive structural elements of the surgical instrument that had to be insulated from each other or via conductors routed centrally in the instrument. Either monopolar or bipolar surgical instruments can be used here.

In the case of monopolar surgical instruments, a single active electrode is arranged on the accessory of the surgical instrument, via which the high-frequency alternating current is introduced into the patient's tissue. In order for the current to be introduced into the tissue from the individual active electrode, a large-area counter electrode (neutral electrode) must be attached to the patient's body as a counter pole.

In the case of bipolar surgical instruments, two electrodes (active electrode and neutral electrode) are attached to the accessory of the surgical instrument. The high-frequency alternating current is introduced into the target tissue from a first electrode (active electrode) directly opposite the second electrode (neutral electrode). In particular, high demands are placed on the insulation of the current-conducting components, for which only thin wall thicknesses are available in the very small installation space available. To avoid voltage flashovers, the applicable voltages are limited accordingly.

Furthermore, previous systems that have a bipolar HF function are sometimes very complex and costly to manufacture and assemble due to the large number of parts. In addition, the mechanical stability of previous bipolar HF instruments is also weaker than that of comparable monopolar or non-active instruments.

### SUMMARY

Against this background, the present invention addresses the problem of providing an improved handling device for electro-surgical instruments, an improved method, a method for producing a handling device for electro-surgical instruments, and a corresponding electro-surgical instrument.

According to the invention, this problem is solved by a handling and/or by a method and/or by an electro-surgical instrument.

Accordingly, the following are provided:
A handling device for an electro-surgical instrument, having: a joint mechanism which has at least one handling-side lever which can be moved for manipulation; a lever extension which is designed as an electrical insulator and is fixedly coupled to the lever; and an electrically conductive contact portion which is fixedly coupled to the lever extension, is arranged on a side of the lever extension which can be subjected to force via the joint mechanism and is designed for electro-surgical contacting of biological tissue.
A method for producing a handling device for an electro-surgical instrument, in particular a handling device according to the invention, having the steps of: Form-fittingly connecting a lever extension designed as an electrical insulator to a lever of a joint mechanism; fixing the form-fitting connection between the lever extension and the lever by means of a securing element; form-fittingly connecting an electrically conductive contact portion designed for electro-surgical contacting of biological tissue to the lever extension on a side which can be subjected to force via the joint mechanism; and fixing the form-fitting connection between the lever extension and the contact portion by means of a securing element.
An electro-surgical instrument, in particular a forceps instrument, with a handling device according to the invention and/or produced using a method according to the invention.

The underlying insight of the present invention is that the alternating current transmission of an electro-surgical instrument can be decoupled from the power transmission so that insulation within the joint mechanism can be dispensed with.

The idea underlying the present invention is to eliminate the previous need to transmit the current via the joint mechanism by integrating the insulator into the lever of the handling device. For this purpose, the lever is designed in several parts, wherein the joint mechanism or its lever is electrically separated from an electrically conductive contact portion by a lever extension designed as an electrical insulator. To prevent the parts from coming loose from each other, the lever extension is firmly connected to the lever of the joint mechanism and the contact portion. Advantageously, the contact portion can be electrically contacted with a conductor directly in this way, independently of the joint mechanism, for example with a conductor passed through the insulator.

In this way, the mechanical and chemical design of the joint mechanism can be advantageously decoupled from the electrical design of the handling device, which allows significantly higher maximum voltages to be used. At the same time, compared to instruments without HF function, no changes to existing joint mechanisms of surgical instruments or their design and in particular their choice of material are necessary. The relatively expensive overmolding of individual parts for insulation, which is often used in other solutions, can also be dispensed with. If biocompatible materials are involved, the mechanically stable design of existing joint mechanism solutions can be used directly in this respect, especially without HF function. Nevertheless, the voltage limitations of an electro-surgical instrument caused by the installation space can be avoided, especially for bipolar operation. In this way, it is possible to modify existing concepts of the joint mechanism of a handling device by means of machining production technology and to supplement them with the lever extension and contact portion according to the invention. This provides an improved handling device for electro-surgical instruments in terms of creep resistance and mechanical load-bearing strength compared to previous solutions.

Since the contact portion is arranged on a side of the lever extension that can be subjected to force via the joint mechanism, the lever extension, which is designed as an insulator, is predominantly subjected to pressure when force is applied, which leads to a continued high mechanical load-bearing capacity of the handling device. For example, a design of an existing handling device, such as pliers, clamps, scissors or the like, which are usually made of corrosion-resistant steel, can be largely adopted in the area of the joint mechanism and only modified on the handling side, i.e., in the area of the distal lever ends, with a lever extension according to the invention and a contact portion according to the invention.

In principle, electrically insulating biocompatible materials, such as plastics, ceramics or the like, can be considered for the lever extension designed as an electrical insulator. Preferably, the lever extension contains at least one mechanically strong and creep-resistant material that fulfills both the electrical requirements for an insulator and the mechanical requirements for the relevant load case of a handling lever for surgical instruments.

The handling device can have a fixed or movable counterpart to the lever, which is designed to take up forces transmitted via the contact portion. In particular, a second lever that can also be moved for manipulation can be provided.

The contact portion can be made of electrically conductive, biocompatible materials such as steel, titanium or similar. The contact portion is designed, for example, as a mechanical gripping or clamping surface or as a blade for biological material. It can be provided with a suitable surface structure for this purpose.

The contact portion forms the active handling surface of the handling device, for example the gripping surface of the forceps in the case of forceps. The rest of the electro-surgical instrument forms an electrically neutral frame that realizes the movements for displacement, rotation or force application.

The design of a handling device according to the invention with a compact format and improved insulation allows it to be used in particular for bipolar HF surgery with comparatively high operating voltages. This qualifies the handling device according to the invention both for manually operated surgical instruments and for use with instruments in surgical systems or with surgical robots. In both cases, these may be endoscopic instruments in particular.

The method according to the invention provides for form-fittingly connecting the lever extension to the lever and for form-fittingly connecting the contact portion to the lever extension. In particular, the lever, the lever extension and the contact element are specially shaped for this type of connection, i.e., designed with corresponding fitting surfaces. In addition, an integrally bonded connection, such as adhesive bonding and/or at least partial welding, can also be provided. The form-fitting connection with the securing element provides a particularly fail-safe connection. This ensures a very high level of operational safety despite the multi-part nature of the handling device.

Advantageous embodiments and developments are shown in the further dependent claims and in the description with reference to the figures in the drawing.

According to one embodiment, the lever extension is form-fittingly connected to the lever. For this purpose, the lever extension and the lever interlock mechanically in such a way that they cannot separate from each other. In particular, the mechanical connection means that the lever extension and the lever cannot come loose from each other, even without or with interrupted power transmission. This provides the advantage of a permanently fixed connection that offers a very high level of security. In addition to the form-fitting connection, an integrally bonded connection, such as adhesive bonding, can also be provided.

According to one embodiment, the form-fitting connection has a circumferential tongue-and-groove connection around a handling-side end of the lever and a corresponding mounting portion of the lever extension. The handling-side end corresponds in particular to a distal end. For this purpose, the lever extension and the lever engage with each other around the handling-side end. Furthermore, perpendicularly or obliquely aligned edge surfaces can be provided on the handling-side end and on the mounting portion so that contact surfaces aligned in different directions are provided, on which normal forces act under load, so that the contact surfaces block translational and, in interaction, also rotational degrees of freedom. Surface pressure remains comparatively low here, so that a very stable connection, in particular a rigid connection with high strength, is advantageously provided. The shape of the tongue and groove is customizable. This means that, in addition to a flat design, which results in a so-called T-slot connection in the cross-section in particular, angled designs in the form of a dovetail connection or even curved shapes are also conceivable. In addition to the form-fitting connection, a frictionally engaged connection can also be provided. In particular, it can be a snap-in or click connection between tongue and groove. Furthermore, in addition to the form-fitting connection, an integrally bonded connection, in particular adhesive bonding, can be provided between the lever extension and the lever.

According to one embodiment, the tongue-and-groove connection is fixed with an additional locking mechanism. In particular, this locks the direction in which the lever extension and the lever are joined together. In particular, this is the insertion direction, that is to say the direction in which the tongue is inserted into the groove. The locking device is in particular a securing element that can be connected to the lever with an integrally bonded connection, such as a pin, rivet or similar. The locking mechanism blocks the direction of insertion and is fixed in at least one of the connecting partners. For example, a pin can be inserted into a recess, in particular a hole, extending into the lever and the lever extension and can be welded to the lever as a securing element.

According to one embodiment, the contact portion is form-fittingly connected to the lever extension. For this purpose, the lever extension and the contact portion engage with each other mechanically in such a way that they cannot separate from each other. In particular, the mechanical connection means that the lever extension and the contact portion cannot come loose from each other, even without or with interrupted power transmission. This also provides the advantage here of a fixed connection that offers a very high level of security. In addition to the form-fitting connection, an integrally bonded connection, such as adhesive bonding, can also be provided. According to a development, the form-fitting connection is also formed here with a circumferential tongue-and-groove connection around a handling-side end of the lever extension and a corresponding mounting portion of the contact portion. The handling-side end corresponds in particular to a distal end. In this way, the lever extension and the contact portion engage with each other around the handling-side end. Furthermore, edge surfaces aligned vertically or at an angle can be provided on the handling-side end and on the mounting portion. As with the form-fitting connection with the lever, this locks translational and, in combination, rotational degrees of freedom and provides a very stable connection. In addition to the form-fitting connection, an integrally bonded connection can also be provided between the lever extension and the contact portion, in particular it can be an adhesively bonded connection.

According to one embodiment, the tongue-and-groove connection between the lever extension and the contact portion can be fixed with an additional locking mechanism. In particular, a securing element connectable to the contact portion with an integral bond is provided for this purpose. The locking mechanism locks the direction in which the lever extension and the contact portion are joined together. In particular, this is the insertion direction, in which the tongue is inserted into the groove. The securing element is in particular a plate provided with an extension, for example a pin, which is connected to the contact portion by an integral bond, in particular welded, and blocks a relative movement in the insertion direction.

According to one embodiment, the lever extension mechanically extends the lever. This means that the lever extension, especially on its own, forms a portion of the effective lever arm. The lever extension is thus designed as a functionally integral component which, in addition to electrical insulation and pressure force transmission, also transmits a torque around the lever axis.

According to a development, the contact portion is arranged in an area that is mechanically extended by the lever extension and into which the lever does not extend or at least does not extend completely. In this way, a greater insulating wall thickness of the lever extension can be provided, so that there is an increased creepage distance and thus improved flashover protection, which allows higher operating voltages.

According to one embodiment, the lever extension has a passage opening which is designed for an electrical conductor to pass through from the side of the joint mechanism to the contact portion for high-frequency activation of the contact portion. In particular, the conductor, for example in the form of a cable, is guided from the proximal side through the lever extension distally to the contact portion. This means that an electrical connection of the contact portion in the lever extension is advantageously retractable and can be sealed off from the environment. This also enables a direct power connection of the contact portion.

According to one embodiment, the joint mechanism has a second lever that can be moved for manipulation. The second lever forms a mechanical counterpart to the first lever and can, in particular, be constructed symmetrically to the first lever, i.e., have a corresponding second lever extension and a corresponding second contact portion. In this way, forces are advantageously applied symmetrically and reaction forces are mutually compensated.

According to one embodiment, the joint mechanism is designed as a non-emerging joint mechanism so that the levers can be manipulated rotationally without displacement. In particular, the levers can be rotated via a traction element, for example an actuating cord of a surgical instrument. The advantage of this is that it prevents joint portions from protruding during lever movement, into which tissue could become trapped.

According to one embodiment, the second lever is fixedly coupled to a second lever extension designed as an electrical insulator. The second lever extension is also coupled to an electrically conductive second contact portion. The two contact portions are arranged to correspond to each other on the inside of the lever extensions and are designed for bipolar high-frequency treatment of biological tissue. In particular, the two levers, lever extensions and contact portions are symmetrical to each other and can be actuated synchronously. This ensures even force transmission and symmetrical load distribution, enabling a high mechanical load-bearing capacity. The handling device is designed, for example, as a distal accessory of a forceps instrument, wherein the levers, lever extensions and contact elements together form the jaw parts.

According to one embodiment of the electro-surgical instrument, in particular an endoscopic instrument, for example a forceps instrument, the instrument and the handling device are designed for bipolar high-frequency treatment.

The above embodiments and developments can be combined with each other as desired, if appropriate. In particular, all features of the handling device are transferable to the production process, and vice versa.

Further possible embodiments, developments and implementations of the invention also include combinations, that are not explicitly mentioned, of features of the invention described above or below with respect to the exemplary embodiments. In particular, a person skilled in the art will also add individual aspects as improvements or additions to the particular basic form of the present invention.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic longitudinal sectional view of a handling device;

FIG. 2 is an exploded view of a handling device according to a further embodiment;

FIG. 3 is a side view of the handling device according to FIG. 2 in the assembled state;

FIG. 4 is a longitudinal sectional view of the handling device according to FIG. 3;

FIG. 5 is a top view of the handling device according to FIG. 2 to 4;

FIG. 6 is a cross-sectional view of the handling device according to FIG. 5 in area A-A;

FIG. 7 is a cross-sectional view of the handling device according to FIG. 5 in area B-B;

FIG. 8 is a perspective view of a handling device according to an embodiment with two levers;

FIG. 9 is a side view of a surgical instrument designed for manual operation;

FIG. 10 is a schematic representation of a surgical system;

FIG. 11 is a top view of a deflection mechanism for an endoscopic instrument;

FIG. 12 is a top view of a deflection mechanism according to a further embodiment;

FIG. 13 is a cross-sectional view of a deflection mechanism according to FIG. 12 in the area of a connecting member;

FIG. 14 is a perspective view of an endoscopic instrument; and

FIG. 15 is a longitudinal sectional view of the endoscopic instrument according to FIG. 14.

### DESCRIPTION OF PREFERRED EMBODIMENTS

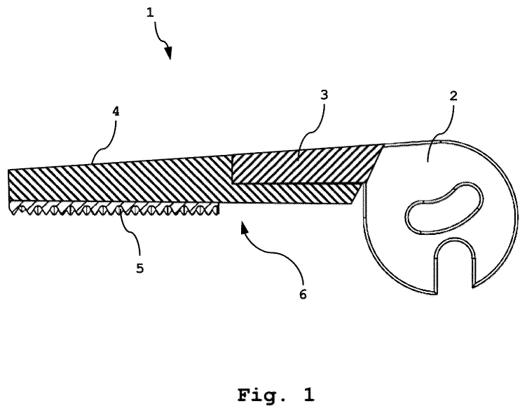

Referring to the drawings, FIG. 1 is a schematic longitudinal sectional view of a handling device 1.

This is a movable handling device 1 for electro-surgical instruments, which can be coupled to a corresponding instrument or its head member, actuating cord and electrical conductor.

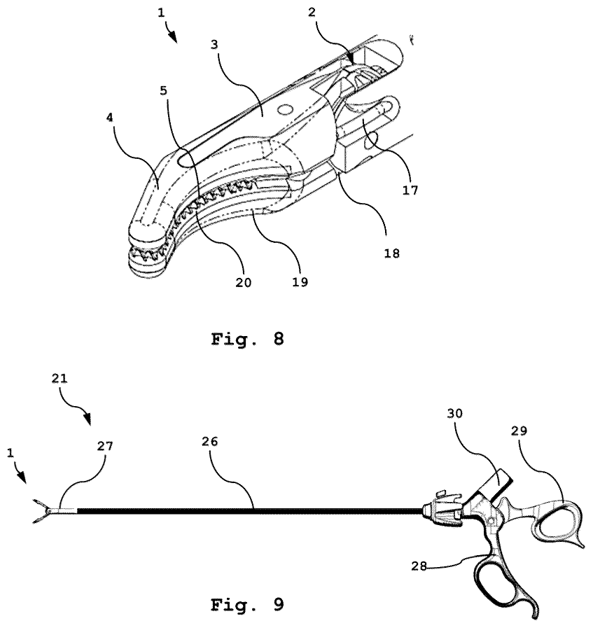

The handling device has a joint mechanism 2, which can be mounted in a head member of an electro-surgical instrument and coupled to an actuating cord. The joint mechanism 2 has at least one handling-side lever 3 which can be moved, in particular rotated, for manipulation by the joint mechanism 2. In addition, the joint mechanism 2 can have a counter-member, not shown here, to absorb the reaction forces of the lever 3 or a second lever, not shown here (see FIG. 8). When mounted, the lever 3 can be rotated by the joint mechanism 2 and can transmit forces, particularly in a closing direction.

The lever 3 is fixedly coupled to a lever extension 4 designed as an electrical insulator. Furthermore, an electrically conductive contact portion 5 fixedly coupled to the lever extension 4 is provided. The lever extension 4 electrically decouples the joint mechanism 2 together with the lever 3 from the contact portion 5, but establishes a fixed mechanical connection between the joint mechanism 2 and the contact portion 5. The lever extension 4 is also designed to transmit force in the closing direction and is subjected to pressure, among other things, during operation of the handling device 1. In the embodiment shown, the lever extension 4 additionally extends the lever 3 and is therefore also subjected to a moment or, depending on the point of application of a reaction force, also to bending.

The contact portion 5 is arranged on a side 6 of the lever extension 4 that can be subjected to a force, in particular the closing force, via the joint mechanism 2. For example, the handling device 1 can be a jaw part of a forceps instrument. In the case of forceps, for example, the side 6 to which force can be applied is the inside of the lever 3.

The contact portion 5 is made of an electrically conductive biocompatible material and has a non-slip and narrow surface contour, which is shown here as an example with corrugation. In this way, the contact portion 5 is designed for electro-surgical contacting of biological tissue. The contact portion can also be contacted directly with a conductor, not shown here, which can extend in particular through the lever extension 4 and be connected to it in an electrically conductive manner on a rear side of the contact portion 5 facing the lever extension 4.

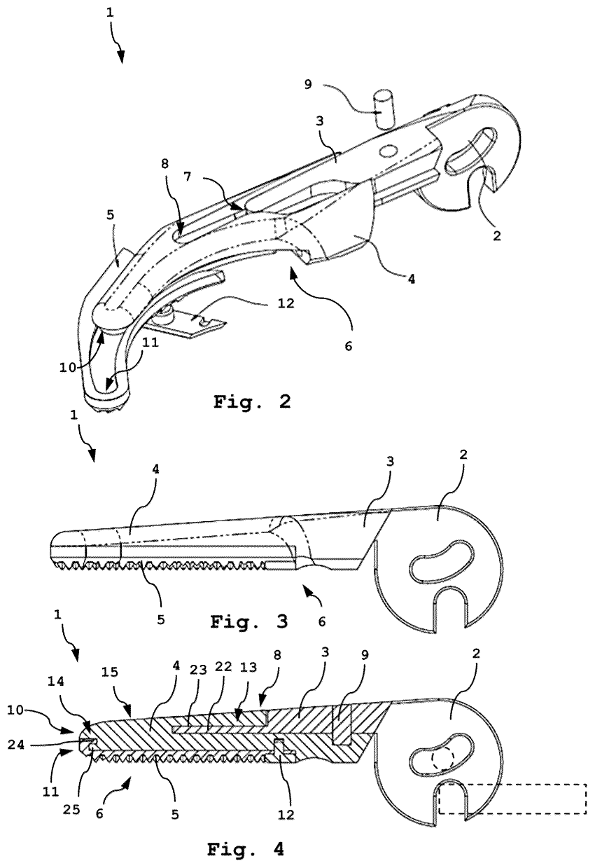

FIG. 2 is an exploded view of a handling device 1 according to a further embodiment.

The features explained in relation to FIG. 1 are transferable to this embodiment. In addition, the lever extension 4 is form-fittingly connected to the lever 3. Furthermore, the contact portion 5 is also form-fittingly connected to the lever extension 4. In the illustrated embodiment, this is a circumferential tongue-and-groove connection 13, 14 by means of so-called T-slots, which engage with each other. For this purpose, a circumferential form-fitting engagement of the tongue-and-groove connection 13 around a handling-side end 7 of the lever 3 is created by sliding on a corresponding mounting portion 8 of the lever extension 4. Similarly, a circumferential form-fitting engagement of the tongue-and-groove connection 14 around a handling-side end 10 of the lever extension 4 is produced by sliding on a corresponding mounting portion 11 of the contact portion 5. Of course, other geometries of the form-fitting connection are also conceivable in other embodiments, for example with dovetail grooves or the like.

Both the respective components and their contact surfaces taper towards the handling side or towards a distal end, so that a complete form fit is achieved when they are fully pushed on. The tongue-and-groove connections 13, 14 are each secured in this position with an additional locking device 9, 12. Between the lever 3 and the lever extension 4 there is a securing element 9 that can be connected to the lever 3 with an integrally bonded connection, which in the illustrated embodiment is designed as a securing peg or pin that engages through the lever into the lever extension 4 and is welded or soldered to the lever 3. The connection between contact portion 5 and lever extension 4 is fixed with a securing element 12 that can be connected to the contact portion 5 with an integrally bonded connection, which is designed here as a securing plate formed with an extension and attached flat from the inner side 6.

Since the lever extension 4, which is designed as an electrical insulator, largely encloses the lever 3, the lever 3 or the joint mechanism 2 and the contact portion 5 are separated from each other with sufficient creepage distances. If the contact portion 5 makes direct contact with a conductor (17, see FIG. 8), the joint mechanism 2 can therefore be advantageously made of steel without itself acting as a conductor. This means that there is no risk of unintentional power surges.

FIG. 3 is a side view of the handling device according to FIG. 2 in the assembled state.

For assembly, the components are preferably glued together over their entire surface in addition to the form-fitting connection. In this way, the strength of the connection is further increased and joints between the components are filled and completely sealed.

FIG. 4 is a longitudinal sectional view of the handling device according to FIG. 3.

This view clearly shows the two tongue-and-groove connections 13, 14, which are each designed with flat tongues and grooves as examples. In the tongue-and-groove connection 13, a comparatively long tongue 22, which is in one piece with the lever 3, is accommodated in a groove 23 provided in the mounting portion 8 of the lever extension 4. In the tongue-and-groove connection 14 between contact portion 5 and lever extension 4, on the other hand, tongues 24, 25, which are provided on the handling-side end 10 and on the mounting portion 11 and are each designed to correspond to one another and are formed with a hook-like extension, and the grooves resulting from the hook-like shape directly adjoining the tongues 24, 25 engage in one another.

The lever extension 4 mechanically extends the lever 3 starting from the joint mechanism 2 or an axis of rotation symbolized here purely schematically as a dashed line, so that a mechanically extended area 15 of the lever extension 4 extending beyond the lever 3 is provided. In this way, the contact portion 5 is nevertheless arranged in a force-transmitting manner in the mechanically extended area 15, into which the lever 3 does not extend, or at least does not extend completely.

A rotation axis, symbolized here schematically with a dashed line, runs in the joint mechanism 2 in a state of the handling device 1 mounted on an electro-surgical instrument. Furthermore, a schematically dashed actuation line of the electro-surgical instrument is coupled to the joint mechanism 2. The joint mechanism 2 is designed as a non-emerging joint mechanism so that the lever 3 can be manipulated rotationally without displacement.

Also clearly visible in this view is the form-fitting connection created by the securing elements 9, 12 that prevent the particular tongue-and-groove connection from loosening. For this purpose, both the pin 9 engages through the lever 3 and the extension of the plate 12 engages through the contact portion 5 in the lever extension 4, which is designed as an electrical insulator.

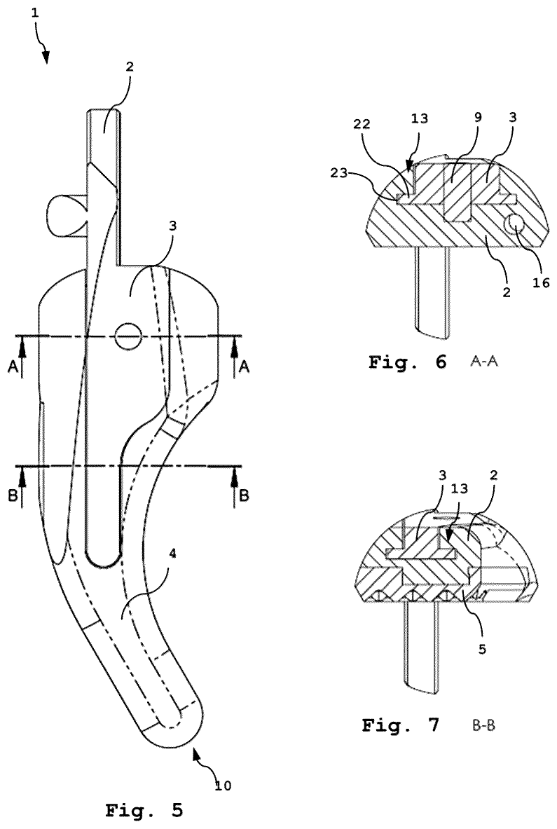

FIG. 5 is a top view of the handling device 1 according to FIGS. 2 to 4.

In this view, it can be seen that the illustrated embodiment of the handling device 1 is a jaw part of surgical forceps. The jaw part has a contour that tapers distally and curves by about 30°. Two intersection lines A-A and B-B are also drawn.

FIG. 6 is a cross-sectional view of the handling device 1 according to FIG. 5 in area A-A.

The sectional line A-A runs transversely through the securing element 9 and illustrates the T-shaped cross-section of the spring 22 of the lever 3 and the groove 23 of the lever extension 4; a passage opening 16 can also be seen, which is designed for an electrical conductor 17 to pass through from the side of the joint mechanism 2 to the contact portion 5 for high-frequency activation of the contact portion 5.

FIG. 7 is a cross-sectional view of the handling device 1 according to FIG. 5 in area B-B.

The intersection line B-B runs through the lever 3, the lever extension 4 and the contact portion 5. The tongue-and-groove connection 13 between the lever and the lever extension is also T-shaped in cross-section here, while there is still no tongue-and-groove connection 14 between the lever extension 4 and the contact portion 5 in this area. This is formed further distally closer to the handling-side end 10.

FIG. 8 is a perspective view of a handling device 1 according to one embodiment with two levers 3, 18.

Accordingly, the joint mechanism 2 has a second lever 18, which can be moved, in particular rotated, in the same way as the first lever 3 for manipulation. The second lever 18 can also be coupled to the actuating cord of an electro-surgical instrument via the joint mechanism 2. In this case, the two levers 3, 18 act together, preferably synchronously, to transmit force. This means that biological tissue can be clamped in between or severed.

Furthermore, the second lever 18 is fixedly coupled in the same way as the first lever 3 to a second lever extension 19 designed as an electrical insulator, which in turn is coupled to an electrically conductive second contact portion 20. The two contact portions 5, 20 are arranged correspondingly to each other on an inner side of the lever extensions 4, 19 and can each be electrically contacted independently of each other with a conductor 17.

The lever extensions 4, 19, which are designed as electrical insulators, enclose the respective levers 3, 18 and electrically separate the particular contact portion 5, 20 from it, so that sufficient creepage distances are available for electrical insulation. Each contact portion 5, 20 can therefore be supplied with an electrical potential independently via the conductors 17. The conductors are routed proximally and can be connected to an HF generator there. The connection of the conductors 17 to the contact portions 5, 20 is realized, for example, by a welded connection. In this way, bipolar operation of the handling device 1 is possible with the corresponding alternating current application with two electrical potentials.

The use of such a handling device 1 for bipolar HF surgery is possible both with hand-operated surgical instruments and with surgical or endoscopic instruments for a surgical system, in particular for operation via a surgical robot.

FIG. 9 is a side view of a surgical instrument 21 designed for manual operation.

It is therefore a hand-guided and hand-operated surgical instrument 21, which is exemplified here as a forceps instrument. The surgical instrument comprises an actuating strand extending in a tubular shaft 26 as a traction element (not shown here). The handling device 1 according to FIG. 8 is mounted here as an accessory or tool at the distal end (facing away from a user/surgeon) of the tubular shaft 26 in a head member 27 of the surgical instrument 21. At the proximal end (facing a user/surgeon), a handle 28 and a movable handle limb 29 are provided for actuating the actuating cord.

The surgical instrument 21 is designed for bipolar high-frequency treatment and to this end has a bipolar power connection 30, for example for a coaxial cable. For contacting from the bipolar power connection to the contact portions 5, 20, for example, two conductors 17 extending in or over the tubular shaft 11 to the contact portions 5, 20 of the handling device 1 can be provided. In further embodiments, other types of contact are also conceivable, at least from the power connection to the head member, for example directly via the tubular shaft 26 and/or the actuating cord.

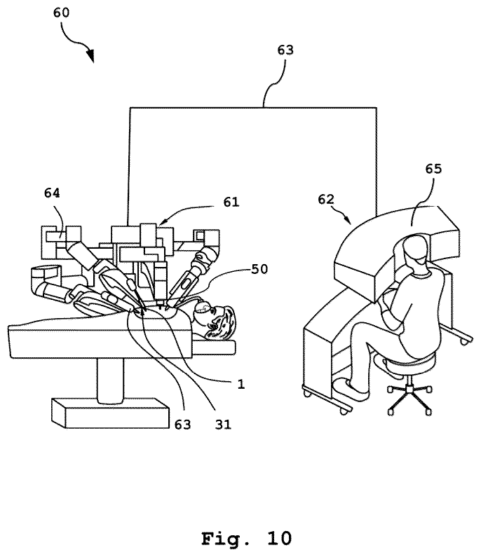

FIG. 10 shows a schematic representation of a surgical system 60.

The surgical system 60 comprises a surgical robot 61 and a corresponding electro-surgical instrument, which is designed as an endoscopic instrument 50.

The endoscopic instrument 50 is guided by means of an arm 64 of the surgical robot 61 and has a handling device 1 which can also be actuated both mechanically and electrically, preferably in a bipolar manner, by means of the surgical robot 61.

Furthermore, the surgical system 60 comprises a control unit 62 connected to the surgical robot 61 via a data connection 63. The control unit 62 is set up to control the surgical robot 61. In particular, the control unit 62 has a man-machine interface 65 via which user inputs or control commands can be entered.

The endoscopic instrument 50 can be detachably connected to the robot arm 64, for example for replacement, modification, sterilization or the like.

In the embodiment shown, the surgical robot 61 has several robot arms, for example. Of course, in further embodiments, a single robot arm or another plurality of robot arms can be provided. Furthermore, several surgical robots and/or several control units can also be provided.

Of the robot arms, only the robot arm 64 is provided with a reference sign for the sake of clarity. Similarly, in the embodiment shown, the surgical system 60 comprises multiple endoscopic devices which may be of the same or different types as the single endoscopic instrument 50, which is also provided with a reference sign for better clarity.

By way of example only, the surgical robot 64 has one endoscopic device per robot arm. The several endoscopic devices can be designed at least partially differently from one another and differ, for example, in the type of end effector and/or the mode of operation. Of course, a person skilled in the art would provide and/or adapt the multiple endoscopic devices for various surgical applications in an obvious manner according to their expertise.

For additional mobility of the handling device 1, the endoscopic instrument 50 has a deflection mechanism 31 between the robot arm 64 and the handling device 1.

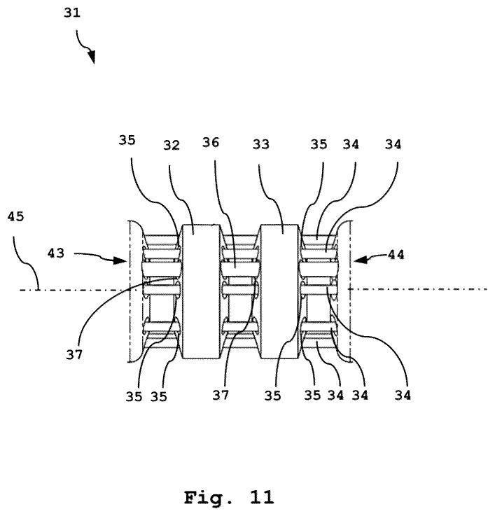

FIG. 11 is a top view of a deflection mechanism 31 for an endoscopic instrument 50.

The deflection mechanism 31 has a first connecting member 32 and a second connecting member 33 which interacts with the first connecting member 32 to deflect it. In further embodiments, even more such connecting members can be provided.

In particular, the connecting members are toroidal or ring-shaped in cross-section around a central axis 45 with a central recess for an actuating cord of an endoscopic instrument. A toroidal shape is to be understood broadly; it can be a ring or an annular disk with a variety of longitudinal section geometries, in particular also with a straight lateral surface in sections and/or a longitudinal section contour that tapers from the inside to the outside.

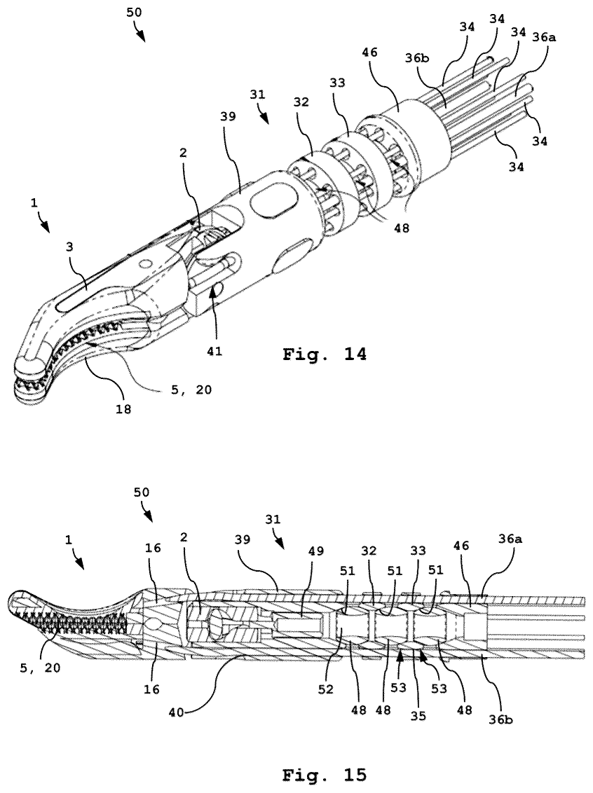

The connecting members 32, 33 are coupled together by a plurality of steering wires 34. In addition, movable coupling elements (48, see FIG. 15) can also be provided in an area of the edges of the central recesses. The steering wires 34 are guided through passage openings 35 in the connecting members 32, 33, which are provided off-center in the connecting members 32, 33. Preferably, the passage openings 35 are arranged at a uniform distance from one another around the center of the connecting members 32, 33.

An electrical conductor 36 is arranged in an area between two adjacent steering wires 24 and also extends, in particular parallel to the steering wires 34, through the connecting members 32, 33. For this purpose, the electrical conductor 36 is guided through additional conductor bushings 37, which are provided in the connecting members 32, 33 between two adjacent passage openings 35 and which are also arranged off-center.

The steering wires 34 are anchored at a distal end 43 of the deflection mechanism 31 in an element distally adjoining the connecting members 32, 33 and can be subjected to tension and/or compression at a proximal end 44 of the deflection mechanism 31, for example by an actuator provided in a robot arm. By applying a tensile load to a steering wire 34, the deflection mechanism 31 is bent towards the steering wire 34 under tensile load. In this way, the distal end 43 is deflected relative to the proximal end 44 in a radial direction, i.e., transversely to a central axis 45 of the connecting members 32, 33.

The at least one conductor 36 moves here automatically with the deflection mechanism 31 and is thus bent comparatively gently in a similar way to the steering wires 340. In addition, the conductor bushings 37 can be designed with inlet and outlet radii in order to avoid edge contact of the at least one conductor 36 and thus in particular so as not to damage the insulation of the conductor 36. In addition, certain compensation volumes can be provided when the conductor 36 is compressed or stretched and the radii of curvature or deformation of the conductor can be kept as large as possible. In this way, despite the free movement of the deflection mechanism 31 in all spatial directions, gentle conductor guidance is always possible, with little mechanical stress on the at least one conductor 36.

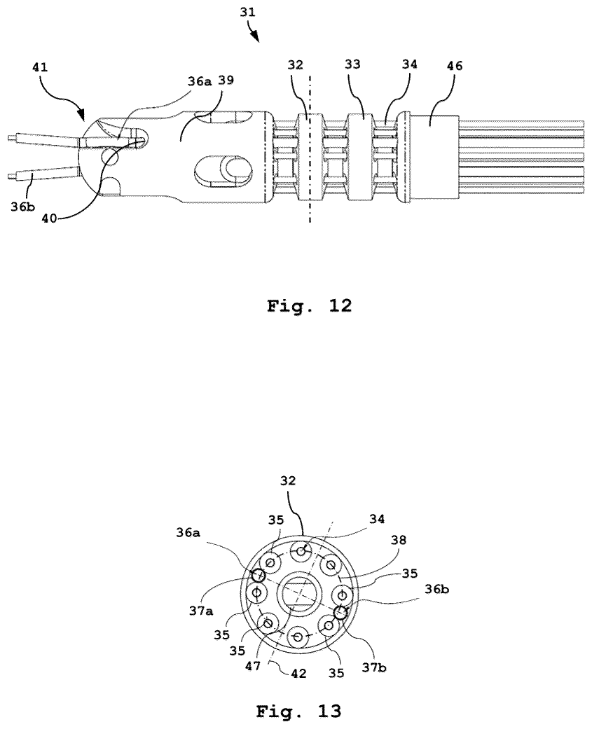

FIG. 12 is a top view of a deflection mechanism 31 according to a further embodiment.

The deflection mechanism 31 also has the features described with reference to FIG. 11, wherein the steering wires 34 are anchored here in a head member 39 which can be coupled to a handling device 1 (see FIG. 14) and which is provided at the distal end 43 of the deflection mechanism 31.

Furthermore, the deflection mechanism 31 here has two electrical conductors 36a, 36b which extend through the connecting members 32, 33 and which are guided in parallel through the connecting members 32, 33. Like the conductor 36 in FIG. 11, both conductors 36a, 36b are each guided in conductor bushings 37a, 37b provided off-center in the connecting members 32, 33 and the conductor bushings 37a, 37b are each arranged in an area between two adjacent passage openings 35. In the area of the head member, the conductors 36a, 36b are guided through a conductor bushing 40 provided in the head member 39 to a head member end 41 and can thus be coupled to a handling device 1. At the head member end 41, the conductors 36a, 36b are guided laterally through the conductor bushings 40 past the joint mechanism 2. The conductor bushings 40 open distally like a funnel so that there is enough space for the conductors 36a, 36b to move when the levers 3, 18 are opened and closed.

At the proximal end 44 of the deflection mechanism, a foot member 46 is provided which can be coupled, for example, via a tubular shaft or directly to a robot arm and which has passage openings for the steering wires 34 and conductor bushings for the conductors 36a, 36b arranged in the same way as in the connecting members 32, 33.

FIG. 13 is a cross-sectional view of a deflection mechanism 31 according to FIG. 12 in the area of a connecting member 32.

As an example, FIG. 12 shows a sectional line through the first connecting member 32, in which the cross-section is arranged. Equally, however, the second connecting member 33 can also have the same cross-section.

As can be seen in the sectional view, the through-openings 35 are arranged here on a concentric pitch circle 38 of the connecting members 32, 33. For reasons of symmetry, it is advantageous to provide an even number of steering wires 34, for example eight in the embodiment shown, evenly distributed over the pitch circle 38 and a corresponding even number of passage openings 35, also for example eight here. With an even number, which in particular is at least eight, a uniform circular deflection of the deflection mechanism without preferred directions, i.e., equally in all directions, is possible. Accordingly, the connecting members 32, 34 are each axially symmetrical in cross-section with respect to a transverse axis 42.

The conductor bushings 37 are also arranged symmetrically, in this case opposite each other, in the area of the pitch circle 38. In the embodiment shown, the conductor bushing 37 is not arranged with its center point exactly on the pitch circle 38, but radially offset to it. Of course, other arrangements in the area of the pitch circle are also conceivable, wherein a cross-section of the conductor bushing 37 preferably intersects the pitch circle 38.

The recess 47 is arranged centrally in the cross-section for the passage of an actuating cord.

FIG. 14 is a perspective view of an endoscopic instrument 50.

The endoscopic instrument 50 is intended for a surgical system 60 according to FIG. 10 and has a deflection mechanism 31 according to FIG. 12 and a handling device according to FIG. 8, which is coupled to the deflection mechanism 31. For coupling, the joint mechanism 2 of the handling device 1 is mounted in the head member 39 and can be actuated by an actuating cord that can be inserted through the center of the deflection mechanism 31.

The handling device 1 has two levers 3, 18 that can be moved synchronously for manipulation. A contact portion 5, 20 is mechanically coupled to each of the levers 3, 18 in an electrically insulated manner. Furthermore, the conductors 36a, 36b are each independently electrically connected to one of the contact portions 5, 20, so that the surgical instrument 50 is configured for bipolar high-frequency treatment when two different electrical potentials are applied, as described with reference to FIG. 8. An electrical potential can therefore be applied to each contact portion 5, 20 independently via the conductors 36a, 36b. The conductors 36a, 36b are, to this end, routed proximally through the head member 39 and the deflection mechanism 31 and can be connected there to an HF generator.

Accordingly, the endoscopic instrument 50 is designed for bipolar electro-surgical contacting of biological tissue. When an equal electrical potential is applied to the conductors 36a, 36b, however, monopolar operation is also possible, which is made possible in particular by the electrical insulation of the contact portions 5, 20 to the joint mechanism 2 with sufficient creepage distance, as shown with reference to FIGS. 1 and 2.

FIG. 15 is a longitudinal sectional view of the endoscopic instrument 50 according to FIG. 14.

This view also shows the coupling elements 48, which are designed as so-called vortex olives. These have a spherical or dome-like shaping on their outer surface. A corresponding spherical segment-shaped recess or socket is provided at the axial ends of the corresponding receptacles 51 of the connecting members 32, 33, which are shaped here as a socket disk, as well as of the head member 39 and the foot member 46. The first connecting member 32 and the second connecting member 33 thus interact in the manner of a ball joint and/or in the manner of vertebral bodies. In this way, the coupling elements 48 and connecting members 32, 33 can be rotated to a certain extent relative to one another, which enables the deflection of the deflection mechanism 31.

The coupling elements 48 are also formed with a central recess 52 for the passage of an actuating cord. An actuating cord can be fastened in an eyelet 49 anchored in the joint mechanism so that the two levers 3, 18 can be rotated and subjected to a closing force by pulling the actuating cord. For example, the eyelet 49 to this end can have an internal thread and the actuating rod can have a corresponding external thread at its tip. Of course, other, preferably detachable fastening means for coupling the actuating cord are also possible.

Also clearly visible in the longitudinal section are inlet and outlet radii 53 provided in the conductor bushings 37, which keep the radii of curvature or deformation of the conductor as large as possible when the deflection mechanism 31 is deflected.

In addition, the passage openings 16 for the conductors 36a, 36b through the electrically insulated sections, in particular a lever extension 4, from the joint mechanism 2 to the contact portions 5, 20 can also be seen here.

Although the present invention has been fully described above with reference to preferred exemplary embodiments, it is not limited thereto, but can be modified in a variety of ways.

In particular, the handling device 1 can also have a different design as an alternative to a jaw part of a forceps instrument. In particular, a lever extension 4 does not necessarily have to extend the lever 3; it could also simply extend the lever 3 in the direction of force transmission.

Furthermore, it would also be conceivable to form a base of the handling device in one piece as a joint mechanism 2 and lever extension 3 made of an insulator material, so that the joint mechanism is made of the insulator material. Preferably, however, it is a three-part structure with an electrically neutral joint mechanism 2 as the base, a lever extension 4 as the insulator and a contact portion 5 as the electrically active contact surface.

The joint mechanism 2 and the head member 39 supporting the joint mechanism 2 can be made of a metallic material, but are not used for current conduction. An electrical potential on the contact surfaces is carried exclusively by conductors 36 or cables that conduct the current directly to the contact surface.

All connections of the individual parts, in particular miniaturized T-grooves and locking pegs or pins, can also be connected by an adhesive bond. The form-fitting connection is therefore a form-fitting securing of the bonded connections.

Previously known jaw parts, for example according to EP 2 026 705 B1, can be modified by dividing them in the manner according to the invention. In particular, the joint mechanics of the non-emergent joints can be retained. The contact surface of the jaw part formed by the contact portion 5 is electrically decoupled from the base or joint mechanism 2 by the lever extension 4. The contact surface of the contact portion 5 thus forms the active gripping surface of the forceps. This structure is analogous to the other jaw part. The rest of the instrument forms an electrically neutral frame, which realizes the movements. The lever extension, which is designed as an electrical insulator, is mainly subjected to pressure and therefore has no mechanical weak point. Securing the form-fitting connection by means of integrally bonded, in particular welded-in, locking pins ensures that the individual parts can no longer fall apart.

The deflection mechanism 31 can comprise different numbers of steering wires 34. In particular, the steering wires 34 can be reduced compared to a comparably dimensioned deflection mechanism, for example from 10 to 8 steering wires when the deflection mechanism is dimensioned with a diameter of 10 mm.

The conductor bushings 37 can, for example, be arranged radially around the central axis 45 as cable ducts like the steering wires 34. This offers the advantage that the cables can follow any movements forced by the steering wires 34. Furthermore, the conductor bushing 40 provided in the head member 39 up to a head member end 41 and also the passage openings 16 for the conductors 36a, 36b through the electrically insulated portions from the joint mechanism 2 to the contact portions 5, 20 are arranged in such a way that the cables can be routed through the head member 39 directly to the contact portions 5, 20.

While specific embodiments of the invention have been shown and described in detail to illustrate the application of the principles of the invention, it will be understood that the invention may be embodied otherwise without departing from such principles.

### LIST OF REFERENCE SIGNS

1 Handling device
2 Joint mechanism
3 Lever
4 Lever extension
5 Contact portion
6 Side
7 Handling-side end
8 Mounting portion
9 Securing element
10 Handling-side end
11 Mounting portion
12 Securing element
13 Tongue-and-groove connection
14 Tongue-and-groove connection
15 Mechanically extended area
16 Passage opening
17 Electrical conductor
18 Second lever
19 Second lever extension
20 Second contact portion
21 Electro-surgical instrument
22 Tongue
23 Groove
24 Tongue
25 Tongue
26 Tubular shaft
27 Head member
28 Handle
29 Handle limb
30 Power connection
31 Deflection mechanism
32 Connecting member
33 Connecting member
34 Steering wire
35 Passage opening
36 Conductor
36a, b Conductor
37 Conductor bushing
38 Pitch circle
39 Head member
40 Conductor bushing
41 Head member end
42 Transverse axis
43 Distal end
44 Proximal end
45 Central axis
46 Foot member
47 Central recess
48 Coupling element
49 Eyelet
50 Endoscopic instrument
51 Receptacle
52 Recess
53 Inlet and outlet radii
60 Surgical system
61 Surgical robot
62 Control unit
63 Data connection
64 Robot arm
65 Man-machine interface

1. A handling device for an electro-surgical instrument, the handling device comprising:
a joint mechanism which has at least one handling-side lever which is configured to be moved for manipulation;
a lever extension, which is configured as an electrical insulator and which is fixedly coupled to the lever; and
an electrically conductive contact portion, which is fixedly coupled to the lever extension and which is arranged on a side of the lever extension that is configured to be subjected to force via the joint mechanism and is configured for electro-surgical contacting biological tissue.

2. The handling device according to claim 1, wherein the lever extension is form-fittingly connected to the lever.

3. The handling device according to claim 2, wherein in that the form-fitting connection has a circumferential tongue-and-groove connection around a handling-side end of the lever and a corresponding mounting portion of the lever extension.

4. The handling device according to claim 3, wherein the tongue-and-groove connection is secured with an additional locking mechanism.

5. The handling device according to claim 1, wherein the contact portion is form-fittingly connected to the lever extension.

6. The handling device according to claim 5, wherein the form-fitting connection is formed with a tongue-and-groove connection running circumferentially around a handling-side end of the lever extension and a corresponding mounting portion of the contact portion.

7. The handling device according to claim 6, wherein the tongue-and-groove connection is configured to be fixed with an additional locking mechanism.

8. The handling device according to claim 1, wherein the lever extension mechanically extends the lever.

9. The handling device according to claim 8, wherein the contact portion is arranged in a region which is mechanically extended by the lever extension and into which the lever does not extend.

10. The handling device according to claim 1, wherein the lever extension has a passage opening which is configured for an electrical conductor to pass through from the side of the joint mechanism to the contact portion for high-frequency activation of the contact portion.

11. The handling device according to claim 1, wherein the joint mechanism has a second lever which can be moved for manipulation.

12. The handling device according to claim 11, wherein the joint mechanism is configured as a non-emerging joint mechanism, so that the levers can be rotationally manipulated without displacement.

13. The handling device according to claim 11, wherein the second lever is fixedly coupled to a second lever extension configured as an electrical insulator and the second lever extension is coupled to an electrically conductive second contact portion, wherein the contact portions are arranged corresponding to one another in each case on an inner side of the lever extensions and are configured for the bipolar high-frequency treatment of biological tissue.

14. A method for producing a handling device for an electro-surgical instrument, the handling device comprising a joint mechanism which has at least one handling-side lever configured to be moved for manipulation, a lever extension, which is configured as an electrical insulator and which is fixedly coupled to the lever, and an electrically conductive contact portion, which is fixedly coupled to the lever extension and which is arranged on a side of the lever extension that can be subjected to force via the joint mechanism and is configured for electro-surgical contacting of biological tissue, the method comprising the steps of:
form-fittingly connecting the lever extension configured as the electrical insulator to the lever of the joint mechanism;
fixing the form-fitting connection between the lever extension and the lever by means of a securing element;
form-fittingly connecting an electrically conductive contact portion configured for electro-surgical contacting of biological tissue to the lever extension on a side which can be subjected to force via the joint mechanism; and
fixing the form-fitting connection between the lever extension and the contact portion by means of a securing element.

15. An electro-surgical instrument, comprising an endoscopic instrument, having a handling device, the handling device comprising:
a joint mechanism which has at least one handling-side lever which can be moved for manipulation;
a lever extension, which is configured as an electrical insulator and which is fixedly coupled to the lever; and
an electrically conductive contact portion, which is fixedly coupled to the lever extension and which is arranged on a side of the lever extension that can be subjected to force via the joint mechanism and is configured for electro-surgical contacting biological tissue,
wherein the electro-surgical Electro-surgical instrument is produced by the method comprising the steps of:
form-fittingly connecting the lever extension configured as the electrical insulator to the lever of the joint mechanism;
fixing the form-fitting connection between the lever extension and the lever by means of a securing element;
form-fittingly connecting an electrically conductive contact portion configured for electro-surgical contacting of biological tissue to the lever extension on a side which can be subjected to force via the joint mechanism; and
fixing the form-fitting connection between the lever extension and the contact portion by means of a securing element.

16. The handling device according to claim 4, wherein the tongue-and-groove connection is secured with an additional locking mechanism, wherein the securing element is integrally bond connected to the lever.

17. The handling device according to claim 7, wherein the tongue-and-groove connection can be fixed with an additional locking mechanism, wherein the securing element is integrally bond connected to the lever.

18. The handling device according to claim 9, wherein the contact portion is arranged in a region which is mechanically extended by the lever extension and into which the lever does not extend completely.
